(19) United States
(12) Patent Application Publication
(10) Pub. No.: US 2023/0384289 A9
(43) Pub. Date: Nov. 30, 2023

(54) DETERMINING COMPOSITION OF RESPIRATORY AIR

(71) Applicant: Calibre Biometrics Inc., Wellesley Hills, MA (US)

(72) Inventor: Udi E. Meirav, Waban, MA (US)

(73) Assignee: Calibre Biometrics Inc., Wellesley Hills, MA (US)

(21) Appl. No.: 17/985,047

(22) Filed: Nov. 10, 2022

Related U.S. Application Data
(60) Provisional application No. 63/284,112, filed on Nov. 30, 2021.

(51) Int. Cl. G01N 33/497; A61B 5/08; A61B 5/087; A61B 5/097; A61B 5/00

(52) U.S. Cl. CPC G01N 33/497; A61B 5/082; A61B 5/087; A61B 5/097; A61B 5/7267

(57) ABSTRACT

Some embodiments are directed to determining the composition of breath exhaled by a subject. For example, some embodiments are directed to determining a concentration of a gas species in breath exhaled by a human subject, based at least in part upon a measured concentration of the gas species in a chamber which is adapted to hold both breath exhaled by the human subject and ambient air for inhalation by the human subject.

### RELATED APPLICATION

This application claims priority under 35 U.S.C. § 119(e) to U.S. Provisional Patent Application Ser. No. 63/284,112, entitled “COMPOSITION MEASUREMENT AND ANALYSIS OF RESPIRATORY AIR,” filed Nov. 30, 2021, bearing Attorney Docket No. V0340.70007US00, the entirety of which is incorporated herein by reference.

### TECHNICAL FIELD

Embodiments of the present disclosure generally relate to systems, methods and devices for collecting and analyzing respiratory data.

### BACKGROUND

Respiratory air composition can be a useful metric for many applications including medical, health, sports and nutrition. Typically, measuring respiratory air composition is done by collecting exhaled air from the subject directly into a collection tube, or wearing a breathing mask attached to a tube with a directional valve that physically separates exhaled air from inhaled air. The exhaled air is conveyed to an analyzing system configured with sensors that can measure concentration of one or more components of the air, such as oxygen or carbon dioxide.

### BRIEF DESCRIPTION OF DRAWINGS

Various aspects and embodiments of the invention are described herein with reference to the following figures. It should be appreciated that these are schematic figures not necessarily drawn to scale, but rather intended to explain the key features and operating principles of the invention. In the figures:

FIG. 1A is a schematic diagram of a representative flow-through configuration used in accordance with some embodiments of the invention.

FIG. 1B is a schematic diagram of a representative apparatus employing the flow-through configuration depicted in FIG. 1A, in accordance with some embodiments of the invention.

FIG. 2 is a flow chart for a representative method used in accordance with some embodiments of the invention.

FIG. 3 is a flow chart for a representative process of system characterization used in accordance with some embodiments of the invention.

FIG. 4 is a schematic diagram of a representative system incorporated unto a breathing mask in accordance with some embodiments of the invention.

FIG. 5 is a schematic diagram of a representative diffusion configuration used in accordance with some embodiments of the invention.

FIG. 6 is a schematic diagram of a representative partitioned chamber used in accordance with some embodiments of the invention.

FIG. 7 is a flowchart of a representative process for producing input to a computer-implemented procedure for determining the composition of respiratory air, in accordance with some embodiments of the invention.

FIG. 8 is a diagram of an illustrative process for determining the composition of respiratory air using machine learning techniques, in accordance with some embodiments of the invention.

FIG. 9 is a block diagram of an illustrative computer system for use in implementing various aspects of the invention.

### DETAILED DESCRIPTION

The Assignee has appreciated that the need in prior art systems to physically separate exhaled air from inhaled air generally leads to a more cumbersome and intrusive system. As such, the Assignee has appreciated that a method of measuring exhaled air composition that does not require physical separation of exhaled air from inhaled air can significantly simplify respiratory data collection and analysis.

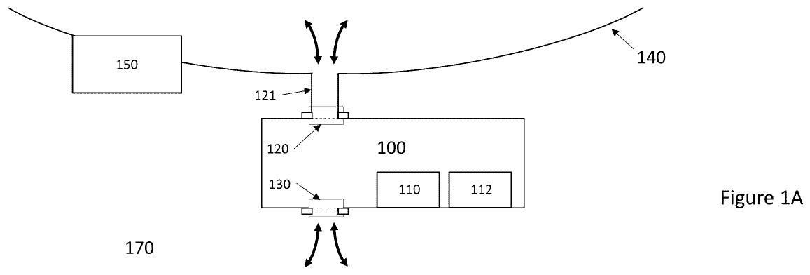

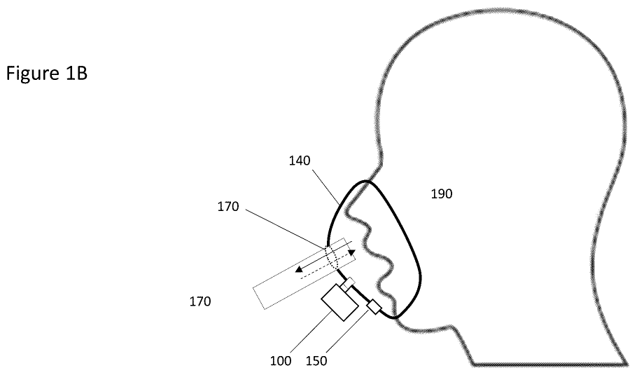

A conceptual illustration of some embodiments of the invention is shown schematically in FIG. 1A, depicting a system for measuring the composition of exhaled breath, comprising a breath sensing subsystem with a sensing chamber or partial enclosure 100 containing an oxygen sensor 110 and a CO2 sensor 112. In some embodiments there are other sensors, not shown in this schematic, that can be incorporated into the sensing chamber. These can be in addition to, or instead of, the oxygen or CO2 sensor shown. The enclosure is further configured with a first aperture 120 providing a path for exhaled air to enter the enclosure. In some embodiments a second aperture 130 provides a flow path to the ambient surrounding, by which exhaled air can exit the enclosure. In some of the descriptions the first aperture 120 may be referred to as inlet, and the second aperture 130 may be referred to as the outlet, although air flow can be in both directions through these apertures. A configuration where air can flow through the chamber by passing via these two apertures in sequence—entering through one and exiting through the other, as shown in FIG. 1A—is referred to herein as a “flow-through configuration”.

FIG. 1A also shows the first aperture 120 connecting to a schematic breath collecting device 140, described in more detail further below. This connection can be a direct aperture in the breath collecting device or can be facilitated by a conduit 121.

In some embodiments the breath collection device 140 is a breathing mask worn on the face of a user, shown schematically in FIG. 1B. The mask can have one or more apertures 170 or tubes (not shown) that create paths for respiratory air flow. These paths need not connect to the sensing subsystem 100.

FIG. 1B shows schematically an embodiment of the system 100 attached to a breathing mask 140, configured to be worn on a subject's face 190. The mask allows inhalation and exhalation through one or more apertures 170. In the embodiment shown, a single aperture 170 is configured to allow part of the respiratory air to flow without having to pass through the system 100. A mask may comprise any suitable configuration and plurality of apertures. The aperture 170 opens directly to the ambient surroundings 180 and allows air to flow in and out, during inhalation and exhalation, respectively. The arrows in FIG. 1B indicate the flow of inhaled (dashed line) and exhaled (solid line) air. There is no separation of inhalation or exhalation flow through different pathways. The respiratory air flow is sensed by a subsystem 150 which will be explained in more detail below. In some embodiments of the invention, a breathing aperture may be attached to a tube or conduit or any other air flow element (not shown). There can be a plurality of such inlets or conduits on a single breathing mask.

It should be emphasized that the schematic illustration of FIG. 1A does not imply or require that all respiratory air flow is conducted through the system 100, only that a fraction of the air flows through the system, whereas the remainder is inhaled and exhaled through alternative flow paths such as the one or more apertures 170 in FIG. 1B.

In other embodiments (not shown) the breath collection device is a tube configured for a user to breathe through, for example by inserting into the mouth or the nose. Any other suitable device or system for collecting, directing or guiding respiratory air flow can be implemented as part of this invention, as long as at least some of the expired air is allowed to enter the system 100.

In some embodiments the system is further configured with a subsystem 150 for sensing respiratory air flow. The sensing can be based on any suitable measurement principle and sensor type. In some embodiments the flow sensor is a pressure sensor that detects changes in air pressure between two points, where pressure can be associated with the direction and rate of air flow. With reference to FIGS. 1A and 1B, the flow sensing pressure sensor shown schematically at 150 measures a pressure difference between the inside of the breathing mask 140 and the external or ambient air 180, and the pressure difference can be used to determine the momentary respiratory air flow, in terms of direction and/or magnitude. Such a differential pressure sensor can be positioned in any suitable location with respect to the path of respiratory air flow. In some embodiments differential pressure is measured between two points, one of which is upstream from the sensing chamber during exhalation and the other is downstream. In other embodiments the pressure sensor measures pressure difference between two different points along the flow path of exhaled air that are both upstream from the chamber. In other embodiments the flow sensor uses a rotating turbine, a vane, an anemometer, or a hot-wire sensor to detect air flow. In other embodiments, any other suitable flow sensing mechanism can be used for the purpose of the method described herein.

In some embodiments the flow sensing subsystem 150 has a response time that is fast enough to determine the start time of each inhalation and each exhalation with adequate precision. It will become apparent that, in some embodiments, precise determination of these times can enhance the overall accuracy of the breath composition measurement.

The sensors are connected to one or more electronic circuits that provide electric power to the sensors and receive output readings from each of the sensors. These outputs are further received by one or more microprocessors, where they are stored and analyzed, and from which they can eventually be transmitted to other systems, e.g. via wireless communication.

As the subject exhales, although much of the exhaled air flows through aperture 170, a fraction of the expired air enters the chamber 100 via the inlet aperture 120. This fractional air stream mixes with the air that is already present in the chamber; at the same time, some of the air is forced to exit the chamber via the outlet aperture 130. Conversely, when the subject inhales, the flow is reversed, and air enters the chamber 100 through the outlet aperture 130 and exits through the inlet aperture 120. All these air flows are schematically depicted in FIG. 1A by the double-edged arrows. The result is that during breathing, the air mix in the chamber varies in a cyclical fashion as the air flowing into the chamber intermittently changes in terms of the ratio of exhaled air—namely, air emerging from inside the lungs where it is depleted from oxygen and enriched with CO2—to inhaled air, which has not yet been inside the lungs.

In a typical embodiment the term “ambient air” is synonymous with the “inhaled air” or, more precisely, the air that is supplied for inhalation. However, in some applications the air made available to the subject for inhalation is provided from a controlled source, for example air that is enriched with oxygen, or filtered, or air whose pressure, temperature or humidity is controlled for the subject's comfort. Nevertheless, in this document—unless explicitly stated otherwise—the term “ambient air” will be understood to mean any air supplied to the subject for inhalation, regardless of whether or not it is drawn directly from the ambient surroundings.

The result is that the concentration of oxygen, CO2 and any other respiratory bio-effluents in the sensing chamber varies gradually and cyclically with the breathing cycle. As a result, the sensor reading at any particular time may not represent pure exhaled air but rather a certain mix of exhaled and inhaled air, and in some embodiments the mixing ratio oscillates with the breathing cycle. Furthermore, gas sensors may have relatively slow response times, and in some embodiments the air composition may be changing faster than the sensors response time, making their interpretation even more complicated or ambiguous.

Some embodiments of the invention are directed to a method for determining the concentration Xₑ of a certain component in exhaled air in such a system. The component can be any molecular species including, but not limited to, oxygen, carbon dioxide, water, alcohol, carbon monoxide, ammonia, acetone or other ketones. The corresponding concentration of the same molecular species in ambient air is Xᵢ.

In some embodiments, a representative method comprises a first step of receiving a series of sequential readings from a sensor that measures the concentration of the component in the sensing chamber X_{sc}, collected over a certain time interval, and calculating a mathematical average of these readings. The average value of the series of readings represents the time-averaged value of the concentration <X_{sc}>, and the concentration of Xₑ in exhaled air is determined from <X_{sc}> by a procedure described below. The ambient air value Xᵢ can be measured at the same time, or can be based on previous measurements, or simply based on known ambient air properties. As a non-limiting example, it is generally known that the concentration of oxygen in dry atmospheric air is about 20.95%, and adjustment of this value for humidity is straightforward.

In some embodiments, the calculation of Xₑ from <X_{sc}> comprises a determination of the average ratio of ambient air to exhaled air present in the chamber over a certain time period. It is instructive to consider two types of edge cases, as follows. In certain conditions the air composition in the chamber 100 changes rapidly with each breath cycle, so that during much of the exhalation time the sensor is exposed almost entirely to exhaled air, while during inhalation it is exposed almost entirely to inhaled or ambient air. This will be referred to as “Type A” conditions.

In Type A conditions, if the duration of inhalation is Tᵢ and the duration of exhalation is Tₑ, then the sensor is exposed over time to the two types of air—namely, inhaled and exhaled—at a ratio equal to Tᵢ:Tₑ, which means that <X_{sc}> is a weighted average of Xₑ and Xᵢ. with the relative weights being Tᵢ and Tₑ:

<X_{sc}>=[Tₑ×Xₑ+Tᵢ×Xᵢ]/[Tₑ+Tᵢ]

The notation can be simplified by defining a factor X that is equal to the ratio of inhalation to exhalation times, also known as the I/E Ratio, expressed as X=Tᵢ/Tₑ. With that notation the quantity of interest, Xₑ is:

Xₑ=(1+λ)×<X_{sc}>−λXᵢ (Equation 1)

An embodiment of the method is therefore as follows:

a. use the flow sensor to determine durations of inhalation and exhalation
b. determine the ratio λ of these durations
c. obtain concentration readings and calculate their average <X_{sc}>
d. use (b) and (c) and knowledge of Xᵢ to determine Xₑ from Equation 1.

In some embodiments, the averaging period may extend over more than a single breath cycle. The value of the inhalation/exhalation time ratio λ can then calculated from the cumulative inhalation and exhalation durations during the averaging period.

In situations that are not clearly Type A, the air in the chamber 100 is replaced slowly or partially with each breath. Equation 1 is then modified by replacing the ratio λ with an adjusted value which will be labeled as μ, which can still depend on Tₑ and Tᵢ but is not necessarily just their ratio (λ), and incorporates an adjustment that will be explained below. In this case the equation takes the form

Xₑ=(1+μ)×<X_{sc}>−μXᵢ (Equation 2)

This may, in certain modes of use, be the more general case. The replacement of λ with an adjusted value μ may better represent conditions where the air composition in the sensing chamber is not entirely replaced by exhaled air immediately upon the onset of exhalation or, conversely, by ambient air upon the onset of inhalation. In a nonlimiting example, vigorous breathing that is associated with more rapid or larger pressure swings is more likely to be associated with rapid displacement of the volume of the sensing space whereas the time to displace its contents will be longer under milder, slower breath. In another non-limiting example, the volume of the sensing chamber and the size of the apertures can influence how quickly the contents of the sensing chamber are displaced as the direction of air flow changes.

The other edge case is referred to as “Type B” conditions, where the value of μ used in the calculation is approximately constant μ=1. In this case Equation 2 can be rewritten simply as Xₑ=2<X_{sc}>−Xᵢ. This value can be a useful approximation in several situations. One non-limiting example where μ≈1 (i.e. approximately equal to 1) is when the duration of inhalation and exhalation are similar, Tᵢ≈Tₑ. Another non-limiting example where μ≈1 is where only a fraction of the air in the sensor chamber 100 is displaced during each breath cycle; in other words the replacement time is long relative to the duration of a breath cycle. This can occur, for example, when the apertures are very small, or resistive to air flow, or the volume of the chamber 100 is large.

However, in some embodiments, the value of μ that is used in the calculation is between 1 and λ, which is likely when the time required to displace the air volume of the chamber is not very short (μ=λ) but also not extremely long (μ=1) either. As an illustration, the value of μ can be (1+λ)/2 which is the mid-point between 1 and λ. More generally it can be any weighted average of 1 and λ, which—for example—can be expressed parametrically as (h+λ)/(h+1) where h is positive.

In other embodiments μ is parametrically dependent on one or more “breath parameters”, including (but not limited to) a measured air flow rate, a breath volume, a breathing rate or frequency (e.g. breaths-per-minute), an inhalation time or duration, and an exhalation time or duration.

In certain embodiments of the method, the parametric dependence of μ on the breath parameters can be obtained empirically by applying the system to a mechanical test apparatus that simulates human respiration with a controlled configuration of “breath parameters” and uses a “test gas” source with known composition to simulate exhaled air. The test gas composition may or may not be similar to normal human exhaled breath. For each configuration the value of μ is obtained by comparing the sensor readings to the known concentration of the gas source. A representative procedure is further described below.

In some embodiments, breath flow is parametrized simply by breath volume (BV), namely an amount of air in one breath. The value of μ varies with BV and X so that it is between 1 and λ. As a non-limiting example, when BV is reduced, μ trends closer to 1 and when BV is increased it trends closer to λ. In certain embodiments the reason for such behavior is that the amount of air entering the chamber in a flow-through configuration is generally proportional to the overall respiratory flow rate. When the breath is small, the air is only partially replaced before the direction of flow changes (inhalation to exhalation or vice versa). Since inhalation and exhalation volumes are typically similar, the mix in the chamber is evenly balanced and therefore μ≈1. On the other hand, if the single breath volume is sufficiently large the air is fully replaced during the breath and the sensor reads exhaled values during much of the exhale cycle, and ambient values during the inhale cycle, corresponding to μ≈λ.

In some embodiments, breath flow is parametrized by a combination of the breath rate BR (number of breaths per minute) and breath volume BV (amount of air per breath). In a non-limiting example of this embodiment, the value of μ is expressed as (h+λ)/(h+1), where h depends on several breath parameters, including but not limited to BR and BV. Table 1 depicts an exemplary dependence of h on Breath Rate (BR) and Breath Volume (BV).

TABLE 1

Milliliters Per Breath

| | | | | |
|---|---|---|---|---|
| Value of h | 400 | 800 | 1200 | 1600 |

| | | | | | |
|---|---|---|---|---|---|
| Breaths | 10 | 0.6 | 0.4 | 0.2 | 0.0 |
| Per | 20 | 0.7 | 0.5 | 0.3 | 0.1 |
| Minute | 30 | 0.8 | 0.6 | 0.4 | 0.2 |

In this arrangement, any value of BV and BR within the range of the table can be interpolated from the table. As an illustrative example of interpolation using Table 1, consider measured values of BV=1000 ml and BR=25 bpm. There is no entry with these values, but the value of h can be interpolated from the table as h=½[½(0.3+0.5)+½(0.4+0.6)]=0.45. Using the value h=0.45, the value of can be expressed as =(0.45+λ)/(1.45), and can be calculated for any combination of Tᵢ and Tₑ—which are inhalation and exhalation times, respectively. For example, for λ=Tᵢ/Tₑ=0.5, then μ=(0.45+0.5)/(1.45)=0.95/1.45=0.65. In this example, if the gas component being measured is CO2 and the average sensor reading is <X_{sc}>=2.8%, and if ambient CO2 is negligible, the imputed value of exhaled CO2 is X_{eCO2}=(1+0.65)×2.8%=4.62%.

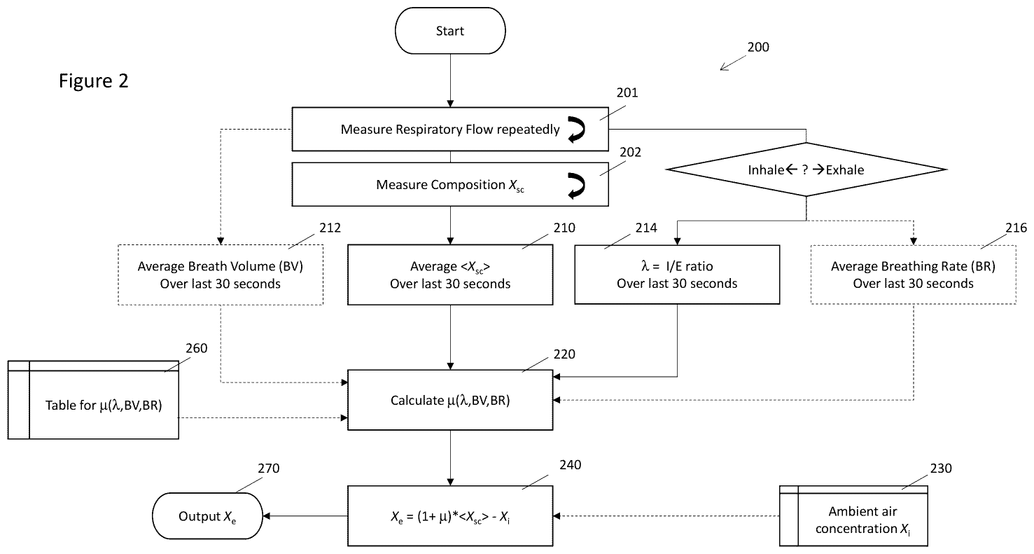

A representative method 200 is depicted in the flow chart of FIG. 2. Respiratory flow data 201 as well as sensors readings from the mixing chamber 202 are collected repeatedly over a certain time duration. The average concentration <X_{sc}> is then computed 210 along with any respiratory flow parameters like λ—the I/E ratio 214—as well as others such as breath volume (BV) 212 and breath rate (BR) 216. In the embodiment of FIG. 2 the averaging is performed over thirty seconds, but in other embodiments any averaging time that is greater, or smaller, than thirty seconds can be used; in other embodiments the averaging time can be a certain number of complete breaths rather than a fixed duration, or a variable controlled by the system user. The averaged values are used in 220 to calculate μ and subsequently in 240 the exhaled concentration Xₑ with the help of the parametric conversion table 260 and a received or known value of ambient concentration 230 is determined and output at 270.

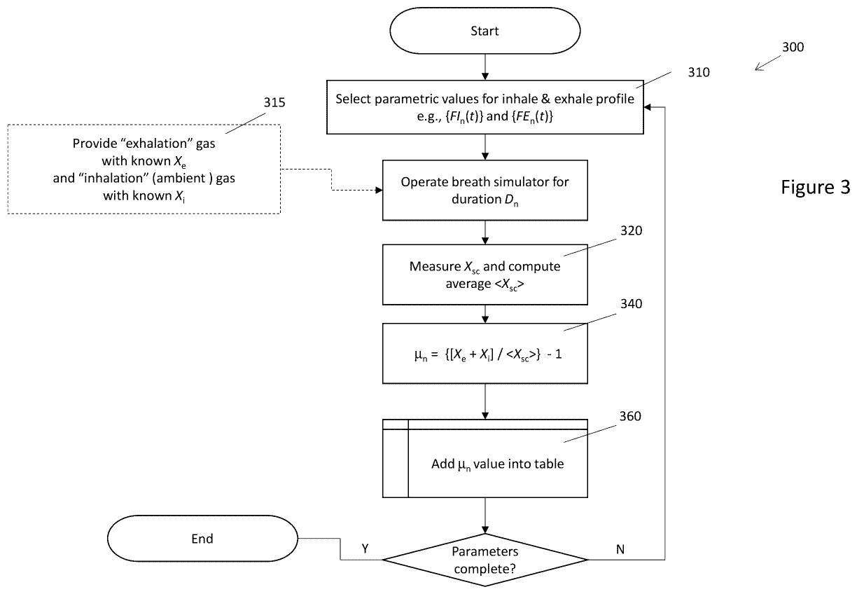

FIG. 3 depicts a representative process 300 for obtaining an empirical conversion table (such as element 260 shown in FIG. 2) corresponding to the properties of a system as described above. The representative process shown includes collecting data from the system while attaching it to a mechanical breath simulator. The breath simulator, shown later herein in more detail, is a mechanical system using gas sources, conduits and flow elements (including, but not limited to, pumps or valves) to control alternating air flows of two gases based on these inputs. The two gases 315 provided to the simulator correspond to (a) exhalation, with known concentration Xₑ (for example, of oxygen and/or CO2) and (b) inhalation or ambient air, with corresponding concentration Xᵢ of the same gas species. The method comprises a sequence of measurements, each performed with a different combination of settings 310 on the breath simulator. Each combination of these settings 310 produces a repeating cyclical air flow pattern, with certain flow rates and durations. Each repetitive cycle may be characterized by a duration and volume of inhalation as well as duration and volume of exhalation; the flow pattern need not be sinusoidal and can have additional characteristics describing the temporal profile of the flow rate over the course of each breath. In one embodiment the simulator is configured to produce certain flow profiles 310—a flow rate vs. time curve—for both inhalation Fᵢ(t) and exhalation Fₑ(t), each using the corresponding gas source and flow direction.

In a sequence of measurements, each measurement can use a different set of parametric settings or profiles, corresponding to a “breath pattern” which is labeled with an index n. In some embodiments each value of n corresponds to a particular breathing pattern, and each breathing pattern is associated with a set of time-dependent flow rates for inhalation FIₙ(t) and for exhalation FEₙ(t). The simulator is controlled to provide the pattern n repeatedly for a duration of Dₙ.

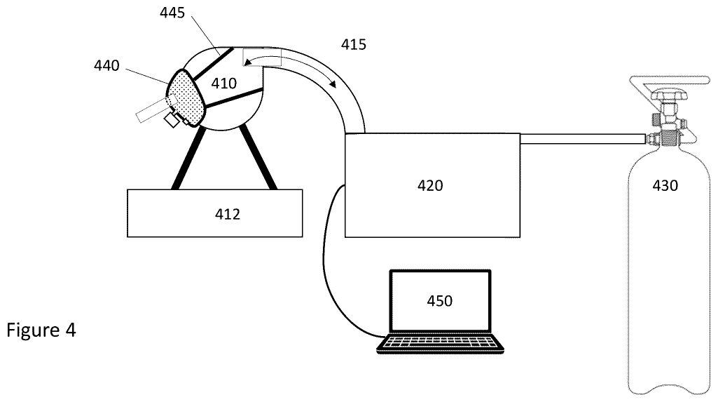

A representative simulator of “breath patterns” is depicted in FIG. 4. The simulator is configured with a mechanical fixture 410, supported by a base 412, approximately simulating a human head, allowing it to attach to the system 440. In the embodiment of FIG. 4, the system 440 is attached to the fixture using a set of head straps 445. The fixture 410 is connected by one or more conduits 415 to a flow management unit 420. The flow management unit comprises components including (but not limited to) any of a pump, bellows, valves, mass flow controllers/sensors, regulators, and a compressor. The system depicted in FIG. 4 further comprises a gas source 430 providing simulated exhaled air, and a computer 450 that controls the direction and rate of gas flow via flow management unit 720 to the fixture. The term “computer” should be understood to include any programmable electronic controller and can connected locally or remotely trough a communications network. During simulated inhalation, the computer instructs the simulator to draw ambient air through from the environment, which is induced to flow through the system 440 (i.e. the mask) and the fixture 410 by a pump in the flow management unit. During simulated exhalation, the computer instructs the simulator to enable gas drawn from the source 430 to flow through the fixture 410 and the system 440. In some embodiment where the gas container 430 is pressurized, a regulator or a mass flow controller, rather than a pump, can be used for “exhalation”. This the programmable computer control of the simulator to produce any desired breath pattern as indicated by 310 in FIG. 3.

The simulator thus provides the intended air flows to the system which, during simulation, measures values of X_{sc} similarly to its normal operation previously described in FIG. 2. During each simulation measurement n the settings 310 are controlled and known by the simulator, while average <X_{sc}> itself is determined 320 by the system attached to the simulator. Using these known values, the next step 340 is to calculate the appropriate value of μ for these parameters. For each simulation n, the inputs 310 and results 340 are collected and stored 360. Interpolation can be used for intermediary values to “fill in” the table which can be used as depicted by 260 in FIG. 2.

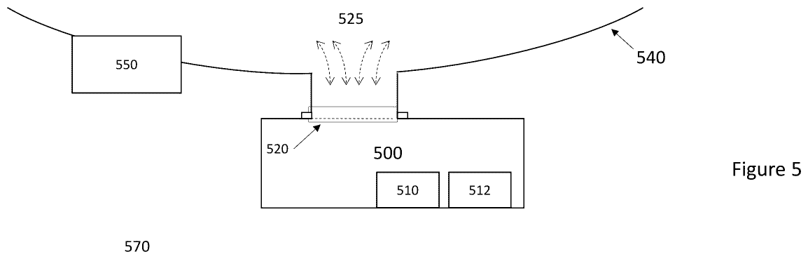

Another representative configuration is shown schematically in FIG. 5. In this embodiment, similarly to the configuration of FIG. 1, a chamber 500 is configured for fluid communication with air in a respiratory collection or flow device 540 such as a mask or a conduit; however, unlike the previous configuration, the chamber is not directly open to the ambient 580 and there is no path for air to flow through the chamber 500 between the collection device 540 and the ambient surrounding 580. Respiratory air 525 enters and exits the subsystem through the aperture 520 from the respiratory flow device predominantly primarily by diffusion or turbulence, and the embodiment depicted is therefore referred to as a “diffusion configuration”. This configuration differs from the “flow-through configuration” shown in FIG. 1A, in which air flow into the subsystem can be facilitated by a static pressure gradient. Despite this difference, both configuration types are similar in that they enable continual mixing of exhaled air with ambient/inhaled air in the space surrounding the gas sensors.

In some embodiments the aperture 520 is protected by an air-permeable screen or filter. The filter may have a benefit of reducing the ingress of unwanted particles, contaminants, water or microorganisms into the chamber or into the sensors.

In some embodiments corresponding to a diffusion configuration, the determination of Xₑ from <X_{sc}> and λᵢ may be different from that of the flow-through configuration. The diffusion configuration may not give rise to significant pressure differences across the aperture 520, so the rate at which air crosses the aperture is less dependent on changing pressures generated by respiration. In these embodiments the exhaled air enters at a relatively constant rate during the entire exhalation cycle, whose duration is Tₑ, and, similarly, ambient air enters at a similar rate throughout the inhalation cycle of duration Tᵢ. Therefore, in some embodiments of diffusion configuration, the average measured concentration <X_{sc}> generally behaves under so-called “Type A” conditions, even if the rate of air diffusion across the aperture is low.

In some embodiments, a system may comprise a plurality of apertures configured to provide fluid communication between the chamber and the collection device 540, but if there are no substantial pressure gradients between these apertures, diffusion remains the primary mechanism of air exchange across these apertures. It will be apparent to a practitioner in the field that multiple apertures can be implemented wherever a single aperture is shown herein.

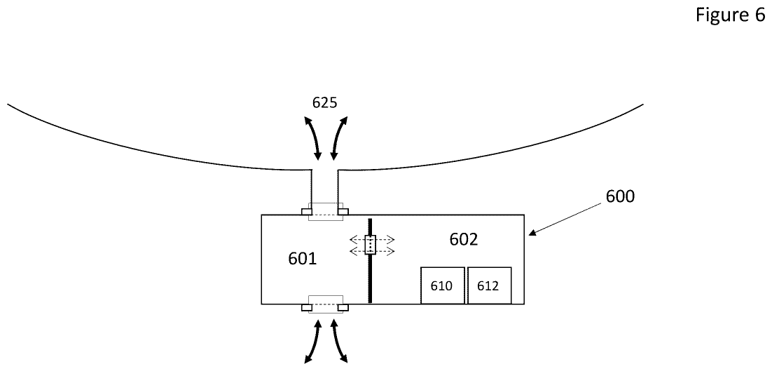

Other suitable configurations of flow paths and apertures are possible. In one non-limiting example, shown schematically in FIG. 6, the subsystem 600 is divided into two spaces: a first space 601 that receives respiratory air from a breath collection system, and a second space 602 where the sensors—shown as 610 and 612—are located. In this configuration the first space 601 can serve as a buffer zone that allows a certain amount of air flow 625 to pass through, while the second space 602 is a sensing zone that receives air primarily by diffusion from the buffer zone 601. The inclusion of such a buffer zone can serve any number of purposes, including but not limited to (a) reducing turbulence near the sensors, (b) protecting sensors from humidity or contaminants, and (c) creating a simple and predictable Type A relationship between <X_{sc}> and λₑ with less sensitivity to other breath parameters. The latter point is explained as follows. If the buffer zone 601 is configured with sufficiently large inlet and outlet apertures, the flow rate through the buffer is relatively high and therefore its content is replaced by newly exhaled air as soon as exhalation begins; the result is that the buffer consists almost entirely of exhaled air throughout the entire exhalation cycle; analogously, it consists almost entirely of ambient air during the inhalation cycle. On the other hand, air continuously enters the sensing zone 602 by diffusion from the buffer zone 601 at all times, and therefore—regardless of its volume and diffusion rate from the buffer—its mixing ratio of exhaled and ambient air does not depend on breath volume, but only the time-average contents of the buffer zone, which leads to Type A conditions and reliable use of Equation 1.

The calculation converting the sensor readings to an imputed value of Xₑ can take any other suitable form. As a matter of principle, detailed and temporally granular measurement of the pressure near the inlet 120, combined with the particular and detailed physical structure of the system, determines the amount of air flowing or diffusing into the chamber at all times, and integration of the flows of ambient and exhaled air over time determines the cumulative mix at any time. This method is reliable as long as the data is sufficiently accurate and the computational power is available to handle the data, while avoiding the complexity, reliability issues and inconvenience of mechanically separating the exhaled air stream from the inhaled air stream. While the foregoing examples provide relatively simple and reliable approximations, these are to be understood as non-limiting examples for the general principle of determining the composition of exhaled air by measuring a mixture of exhaled and inhaled air, and using the recent mixing history to determine, computationally, the corresponding properties of the exhaled air.

The derivation of Xₑ from <X_{sc}> can utilize algorithms with dependency on any number of measured quantities including, but not limited to, multiple readings of air flow rate with high granularity that can be used for imputing the exhaled concentration from the measured concentration of the mixed air. In some embodiments the flow sensing subsystem 150 utilizes commercially available piezo-electric differential pressure sensors, which are available from multiple suppliers including but not limited to Honeywell Inc., Merit Sensor Inc., Robert Bosch GmbH, and Sensirion AG. Several of these commercially-available products can measure pressure, and hence flow, with time resolution as low as 1 millisecond (namely 1000 readings per second). Other types of flow sensing techniques my provide similar or even higher resolution and precision. Thus obtaining 10, 100 or even 1000 readings per second is clearly well within the capabilities of many such devices. In some embodiment the system collects between 1-10 readings per second. In some embodiment the system collects between 10-50 readings per second. In other embodiments the system collects between 50-1000 readings per second.

As a result of these reading rates, the measurements can yield thousands of data points per minute, each corresponding to an instantaneous pressure differential which may directly or indirectly affect the rate of air entering the chamber. The actual mixture in the chamber at any time is a complex but deterministic result of the recent flow and pressure history of the chamber apertures. Thus the concentration X_{sc} being recorded by the air sensor is, in principle, a deterministic result of the exhaled air concentration (Xₑ) and the flow readings over a recent time duration (referred to herein as the Integration Time, or Tᵢₙₜ). The appropriate duration Tᵢₙₜ used for the calculation may depend—among other things—on system design choices and the subject's breath patterns. In some embodiments Tᵢₙₜ is between 10-30 minutes. In some embodiments Tᵢₙₜ is between 1-10 minutes. In some embodiments Tᵢₙₜ is between 30-60 seconds. In some embodiments Tᵢₙₜ is between 5-30 seconds. In some embodiments Tᵢₙₜ is based on the completion of a number Bₙ of breath cycles, rather than a fixed time duration; In some embodiments Bₙ is between 1 and 10. In some embodiments Bₙ is between 10 and 100. In some embodiments Bₙ is greater than 100. In some embodiments Tᵢₙₜ can be determined by any other criteria or variables, including a user preference.

In some embodiments, in order to utilize the relatively large number of readings to determine Xₑ, a system implemented in accordance with some embodiments of the invention may employ one or more computer programs to determine Xₑ, and/or identify the readings or combinations thereof which may be used to reliably determine Xₑ (and/or other values) in a given configuration. Any suitable computational approach(es) may be employed. For example, some embodiments may employ predictive analysis, and use historical data to identify patterns and relationships within a known set of rules. Some embodiments may employ artificial intelligence or machine learning, whereby a computer-implemented system may autonomously test assumptions to grasp insights and identify relationships which are not apparent at the outset.

In some embodiments of this invention, a computer-implemented system may be taught or trained through exposure to a physical breath simulator similar to the one described earlier in FIG. 4. While exposed to the simulator, the system may capture a sequence of values, including but not limited to flow {F} as concentration {X_{sc}} values, produced by a simulator under a variety of simulator settings—namely, breath patterns and compositions. In some embodiments, this exposure may be repeated (e.g., cyclically) as much as needed, to establish fidelity and/or repeatability of results.

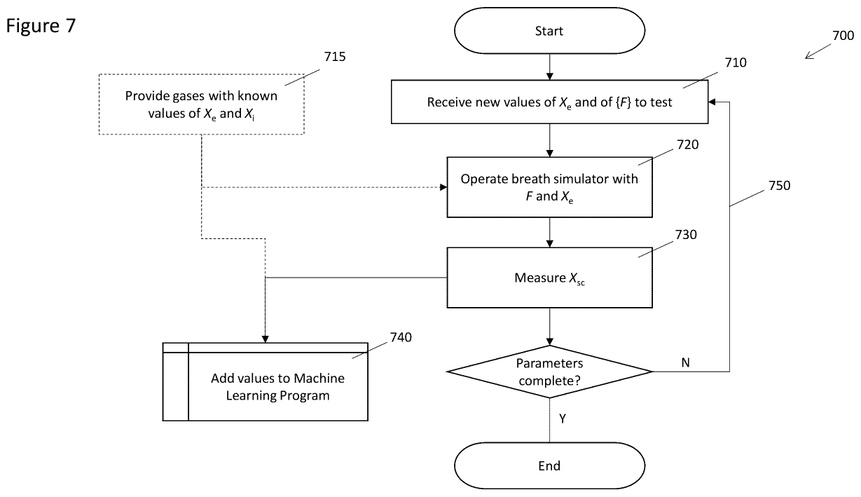

FIG. 7 describes a representative procedure 700 whereby a simulator (such as that of FIG. 4) may produce inputs that can be used to train a computer-implemented process (e.g., a machine learning procedure) for determining one or more values (e.g., Xₑ) and/or identify the readings, measurements, parameters or combinations thereof which may be useful in determining the value(s) in a given configuration. In some ways, the representative procedure shown in FIG. 7 is a generalization of the procedure described earlier in FIG. 3, and comprises a series of iterations where in each iteration a simulated breath pattern is chosen 710; such a breath pattern may comprise a sequence of hundreds or even thousands of sequential values of flow F, collectively {F}, as well as the value of Xₑ. The sequence is then implemented in a repeated fashion on the simulator 720, while the system measures X_{sc} values in 730. After a predetermined time duration, the measured values of X_{sc} can be averaged to obtain <X_{sc}>, or left as a series {X_{sc}}, and the combination of values of F and λ_{sc} are sent to a machine learning database 740, along with the concentration values Xₑ and λᵢ corresponding to the gases provided to the simulator 715. The exercise is repeated 750 with different choice of {F} and λₑ, as many times as required and with as many variation as required.

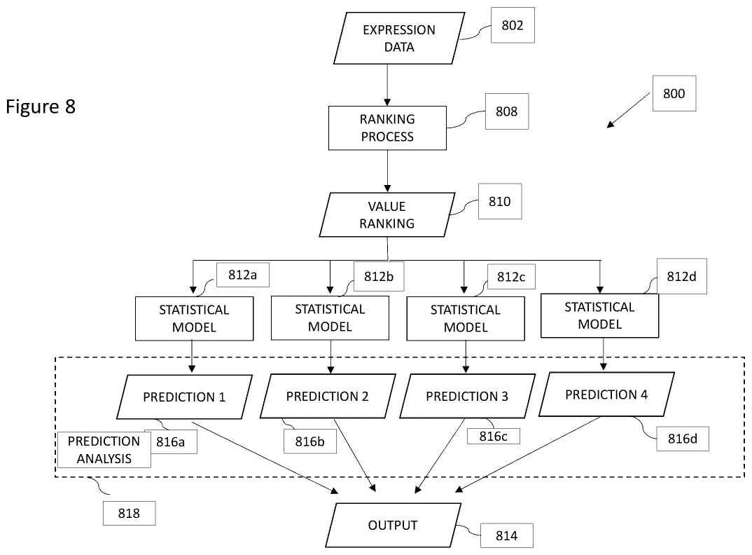

FIG. 8 is a diagram which conceptually illustrates a representative processing pipeline 800 for identifying the value and/or combinations of values (e.g., measurement(s), reading(s), parameter(s), etc.) which are useful in determining Xₑ (as a non-limiting example) in a particular configuration (e.g., a “flow-through” configuration, a “diffusion” configuration, etc.), and/or the extent to which each value and/or combination of values influences Xₑ. In the representative processing pipeline 800 shown, values and/or combinations thereof may be ranked based on their levels of expression in one or more datasets (e.g., machine learning database 740; FIG. 7), and a set of statistical models may then be applied to predict the value or combination of values that is (are) predictive of Xₑ in a particular configuration.

In the example shown in FIG. 8, expression data 802 and ranking process 808 are used to rank values and/or combinations thereof based on their expression levels in expression data 802 to obtain a ranking 810 of values and/or combinations of values. Value ranking 810 is then input to statistical models 812a. 812b, 812c and 812d to generate corresponding predictions. The statistical models 812a-d which are chosen for this purpose may be selected based on any suitable criteria, and may be trained using any suitable training data.

Statistical models 812a, 812b, 812c and 812d may each identify a predicted value or combination of values which are indicative of Xₑ in a particular configuration. In some instances, a prediction output by a statistical model 812 may include a probability and/or an extent to which an identified value or combinations of values is useful in determining Xₑ in a given configuration. As shown in FIG. 8, statistical model 812a outputs Prediction 1 816a, statistical model 812b outputs Prediction 2 816b, statistical model 812c outputs Prediction 3 816c, and statistical model 812d outputs Prediction 4 816d. In the example shown, the predictions produced by statistical models 812a-d are then analyzed using prediction analysis process 818 to identify the one or more values and/or combinations of values which are indicative of Xₑ. Prediction analysis process 818 may involve selecting a particular prediction (e.g., based on an associated probability) from among the different predictions 816a-d to produce output 814, or combining the predictions 816 produced by two or more statistical models 812, in any suitable fashion.

Any of statistical models 812 and prediction analysis process 818 may employ a machine learning algorithm, including one or more classifiers. Examples of classifiers which may be used include gradient boosted decision tree classifiers, decision tree classifiers, gradient boosted classifiers, random forest classifiers, clustering-based classifiers, Bayesian classifiers, Bayesian network classifiers, neural network classifiers, kernel-based classifiers, and support vector machine classifiers. In some embodiments, machine learning algorithm may include a binary classifier, and in some embodiments, a machine learning algorithm may include a multi-class classifier.

It should be appreciated that although FIG. 8 depicts four outputs being produced by four statistical models, a processing pipeline implemented in accordance with embodiments of the invention may include any suitable number of statistical models producing any suitable number of outputs. It should also be appreciated that although processing pipeline 800 is used to predict the values or combinations of values which are useful in determining Xₑ, in a particular configuration, a processing pipeline implemented in accordance with embodiments of the invention may be used to predict any suitable information, as the invention is not limited in this respect.

Additionally, it should be appreciated that once a value or combination of values is identified as being indicative of Xₑ in a particular configuration, the value(s) may thereafter be used to calculate Xₑ in a variety of devices or settings. For example, in some embodiments, an apparatus like that which is depicted in FIG. 1B may include one or more microprocessors programmed to calculate Xₑ in the exhaled breath of a wearer of the apparatus, using one or more previously identified values to perform the calculation. In this respect, it should be appreciated that the value or combination of values which are indicative of Xₑ in a particular configuration may be identified based on measurements, readings, parameters, etc. from a first entity (e.g., a breath simulation apparatus, as shown in FIG. 4), and the value(s) may thereafter be used to calculate Xₑ in the exhaled breath of a second entity (e.g., a human subject, wearing an apparatus like that which is depicted in FIG. 1B).

It should further be appreciated that the term “machine learning” used herein is intended to encompass a wide range of computational approaches, some of which may not conform with a strict definition of the term. For example, as used herein, the term is intended to encompass computational approaches which one skilled in the art may characterize as “artificial intelligence”, “deep learning”, and/or any other form(s) of predictive analysis, whether now known or later-developed.

Processing pipeline 800 may be performed on any suitable computer system (e.g., a single computing device, multiple computing devices co-located in a single physical location or located in multiple physical locations remote from one another, one or more computing devices part of a cloud computing system, etc.), as the technology described herein is not limited in this respect. For example, processing pipeline 800 may be performed on a desktop computer, a laptop computer, and/or a mobile computing device. In some embodiments, processing pipeline 800 may be performed using one or more computing devices which are part of a networked (e.g., cloud) computing environment.

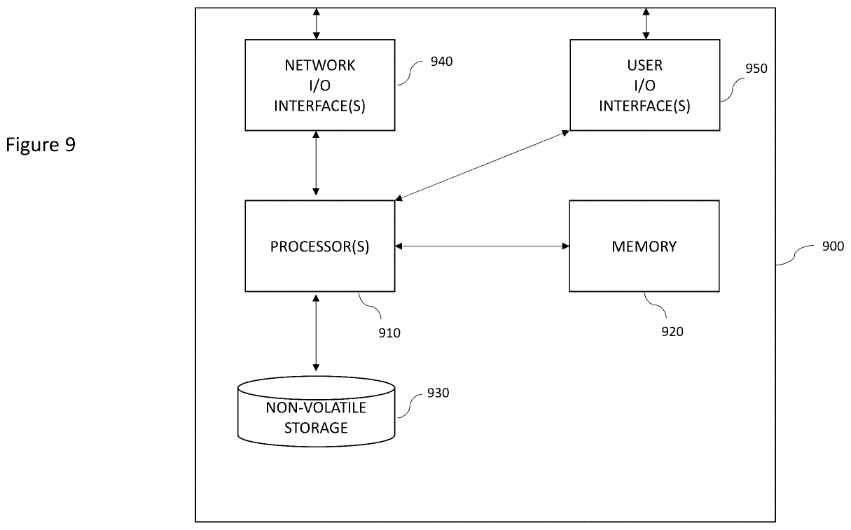

An illustrative implementation of a computer system 900 which may be used in connection with any of the embodiments disclosed herein is shown in FIG. 9. The computer system 900 includes one or more processors 910 and one or more articles of manufacture that comprise non-transitory computer-readable storage media (e.g., memory 920 and one or more non-volatile storage media 930). The processor(s) 910 may control writing data to and reading data from the memory 920 and the non-volatile storage device 930 in any suitable manner, as the aspects of the technology described herein are not limited in this respect. To perform any of the functionality described herein, the processor(s) 910 may execute one or more processor-executable instructions stored in one or more non-transitory computer-readable storage media (e.g., the memory 920), which may serve as non-transitory computer-readable storage media storing processor-executable instructions for execution by the processor(s) 910. The computing device 900 also includes a network input/output (I/O) interface 940 via which the computing device 900 may communicate with other computing devices (e.g., over a network), and one or more user I/O interfaces 950, via which the computing device 900 may provide output to and receive input from a user. The user I/O interfaces 950 may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

Potential Modes Of Implementation. It should be appreciated that while in principle the characterization of a product embodying aspects of the invention can be done once and applied to mass-produced copies of the product, in some embodiments, variations between nominally identical manufactured products are not negligible, e.g. due to manufacturing variations. In these circumstances, individual products may undergo their own customized characterization process using the breath simulator as described above, to achieve maximum accuracy when necessary.

In some embodiments, other variables can be further incorporated in the computational conversion of X_{sc} to λₑ. A few non-limiting examples of these are:
i. The concentration values of other species of gases; for example using measured oxygen concentration in calculating the concentration of CO2 or vice versa.
ii. Environmental sensors such as temperature, humidity and barometric pressure.
iii. Motion and acceleration sensors
iv. Geolocation (GPS)
v. Non-respiratory biometrics—including but not limited to heart rate or blood pressure—measured by including the requisite sensors in the system or by a using a separate device and providing the data to the system as an external electronic input.
vi. Sensors that detect respiratory-related physiological motion, for example motion of the subject's rib cage or facial tissue.

Some of these variables are relatively straightforward to add to mechanical simulation, including but not limited to examples (i)-(ii) above. Other variables maybe more to test with a mechanical breath simulator, but may still be used in other ways to improve computational accuracy.

The further complexity of these additional inputs can also be addressed with machine-learning or other artificial intelligence software algorithms that are implemented in the microprocessors or on an external connected computation system.

By the nature of machine learning and related software techniques, the use of such algorithms to hone the computational conversion of mixture properties to exhaled air properties may lead to superior accuracy but also obscure some of the inner workings of the computational algorithm.

The use of computational algorithms to impute exhaled air properties from mixed air properties—such as X_{sc}—is intended to be considered part of the invention described herein, regardless of whether such algorithms are developed theoretically or empirically (such as by using a breath simulator as described herein), or whether implemented explicitly through software programming or indirectly with the help of machine learning procedure.

It should be appreciated that one mode of implementing embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (e.g., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Generality. Having thus described several aspects of at least one embodiment of this invention, it is to be appreciated that various alterations, modifications, and improvements will readily occur to those skilled in the art. Such alterations, modifications, and improvements are intended to be part of this disclosure and are intended to be within the spirit and scope of the invention. Further, though advantages provided by various embodiments of the present invention are indicated, it should be appreciated that not every embodiment of the invention will include every described advantage. Some embodiments may not implement any features described as advantageous herein and in some instances. Accordingly, the foregoing description and drawings are by way of example only.

Various aspects of the present invention may be used alone, in combination, or in a variety of arrangements not specifically discussed in the embodiments described in the foregoing, and it is, therefore, not limited in its application to the details and arrangement of components set forth in the foregoing description or illustrated in the drawings. For example, aspects described in one embodiment may be combined in any manner with aspects described in other embodiments.

The invention may be embodied as a method, of which various examples have been described. The acts performed as part of the method may be ordered in any suitable way. Accordingly, embodiments may be constructed in which acts are performed in an order different than illustrated, which may include different (e.g., more or less) acts than those which are described, and/or which may involve performing some acts simultaneously, even though the acts are shown as being performed sequentially in the embodiments specifically described above.

Use of ordinal terms such as “first,” “second,” “third,” etc., to modify an element does not by itself connote any priority, precedence, or order of one claim element over another or the temporal order in which acts of a method are performed, but are used merely as labels to distinguish one element having a certain name from another element having the same name (but for use of the ordinal term) to distinguish the elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A system for determining a composition of exhaled breath, the system comprising:
a chamber adapted to, during use by a human subject exhibiting breath cycles, receive air exhaled by the human subject and ambient air for inhalation by the human subject, and thereby hold a variable mix of exhaled and ambient air during the breath cycles;
a gas sensor configured to measure a concentration X_{sc} of a gas species in the chamber;
a subsystem configured to measure respiratory air flow F; and
at least one microprocessor programmed to:
determine, from measurements of X_{sc} by the gas sensor, an average value <X_{sc}>;
determine at least one respiratory value from readings of F by the subsystem;
determine a concentration Xᵢ of the gas species in the ambient air; and
determine, using <X_{sc}>, the at least one respiratory value, and Xᵢ, a concentration Xₑ of the gas species in breath exhaled by the human subject.

2. The system of claim 1, wherein the at least one microprocessor is programmed to determine an adjustment factor μ based at least in part on the at least one respiratory value, and to determine Xₑ according to:
Xₑ=(1+μ)×<X_{sc}>−μXᵢ

3. The system of claim 2, wherein the adjustment factor relates to a ratio of a duration of inhalation Tᵢ to a duration of exhalation Tₑ, expressed as λ=Tᵢ/Tₑ, and the at least one microprocessor is programmed to determine Xₑ according to:
Xₑ=(1+λ)×<X_{sc}>—λXᵢ

4. The system of claim 2, wherein μ=1.

5. The system of claim 1, wherein the at least one respiratory value relates to one or more of a breath volume, a breath duration, a breath rate, a minute volume, an air flow rate, and an inhalation/exhalation ratio.

6. The system of claim 1, wherein the chamber comprises a first aperture and a second aperture, and wherein the chamber is adapted so that, during use by the human subject, exhaled air enters the chamber via the first aperture and ambient air enters the chamber via the second aperture.

7. The system of claim 1, wherein the chamber comprises at least first and second zones, with at least one aperture being located in the first zone and the gas sensor being located in the second zone, the first and second zones being in fluid communication with each other.

8. The system of claim 1, wherein the chamber comprises an aperture, and is adapted so that, during use by the human subject, both exhaled air and ambient air enter the chamber via the aperture.

9. The system of claim 1, wherein the gas species is one of oxygen and carbon dioxide.

10. The system of claim 1, wherein the gas species is one of water, alcohol, ammonia, acetone, and urea.

11. The system of claim 1, wherein the subsystem comprises a differential pressure sensor, an anemometer, or a turbine.

12. A method for determining a composition of exhaled breath, the method being for use in a system comprising a chamber and a gas sensor, the chamber being adapted to, during use by a human subject exhibiting breath cycles, receive air exhaled by the human subject and ambient air for inhalation by the human subject, and thereby hold a variable mix of exhaled and ambient air during the breath cycles, the gas sensor being configured to measure a concentration X_{sc} of a gas species in the chamber, the method comprising acts of:
(A) determining, from measurements of X_{sc} by the gas sensor, an average value <X_{sc}>;
(B) determining a concentration Xᵢ of the gas species in the ambient air; and
(C) determining, using <X_{sc}> and X ᵢ, a concentration Xₑ of the gas species in breath exhaled by the human subject.

13. The method of claim 12, comprising an act of determining at least one respiratory value relating to one or more of a breath volume, a breath duration, a breath rate, a minute volume, an air flow rate, and an inhalation/exhalation ratio, and wherein the act (C) comprises using the at least one respiratory value in determining the concentration Xₑ of the gas species in breath exhaled by the human subject.

14. The method of claim 12, wherein the chamber comprises a first aperture and a second aperture, and wherein the chamber is adapted so that, during use by the human subject, exhaled air enters the chamber via the first aperture and ambient air enters the chamber via the second aperture.

15. A computer-implemented method for identifying one or more values indicative of a composition of exhaled breath, the method comprising acts of:
(A) receiving a plurality of values produced by a system comprising a chamber and a gas sensor, the chamber being adapted to, during breath cycles, receive exhaled air and ambient air, and thereby hold a variable mix of exhaled and ambient air during the breath cycles, the gas sensor being configured to measure a concentration X_{sc} of a gas species in the chamber, the plurality of values comprising:
measurements of X_{sc} by the gas sensor;
a concentration Xᵢ of the gas species in the ambient air; and
data characterizing inhalation or exhalation during the breath cycles; and
(B) identifying certain of the plurality of values as being indicative of a concentration Xₑ of the gas species in exhaled breath.

16. The computer-implemented method of claim 15, wherein the act (B) comprises performing a machine learning process to identify the certain value(s) indicative of Xₑ.

17. The computer-implemented method of claim 16, wherein the act (B) comprises performing a machine learning process to determine an extent to which each of the certain value(s) is indicative of Xₑ.

18. The computer-implemented method of claim 16, wherein the method is for use in the system, the system comprises a breath simulation apparatus configured to produce known or controlled values characterizing inhalation and exhalation during the breath cycles, and the act (B) comprises training the machine learning process based on values produced as a result of operating the breath simulation apparatus.

19. The computer-implemented method of claim 18, wherein the breath simulation apparatus is configured to produce a cyclical or repeating pattern of breath characteristics.

20. The computer-implemented method of claim 15, wherein the act (A) comprises receiving a plurality of values relating to a first entity, and the method comprises an act of:
(C) using the certain value(s) identified in the act (B) to determine a concentration Xₑ of the gas species in breath exhaled by a second entity that is different from the first entity.
